(19) United States
(12) Patent Application Publication
(10) Pub. No.: US 2026/0000276 A1
(43) Pub. Date: Jan. 1, 2026

(54) ENDOSCOPE DEVICE, ADAPTOR AND TUBULAR UNIT OF AN ENDOSCOPE APPARATUS

(71) Applicant: Chang-Bih SHIE, Tainan City (TW)

(72) Inventor: Chang-Bih SHIE, Tainan City (TW)

(21) Appl. No.: 19/245,826

(22) Filed: Jun. 23, 2025

(30) Foreign Application Priority Data
Jun. 28, 2024 (TW) 113124262
Oct. 21, 2024 (TW) 113139918

(51) Int. Cl. A61B 1/00; A61B 1/005; A61J 15/00; A61M 27/00

(52) U.S. Cl. CPC A61B 1/00128; A61B 1/00087; A61B 1/00137; A61B 1/005; A61J 15/0003; A61M 27/00; A61M 2202/0496

(57) ABSTRACT

An endoscope apparatus includes an endoscope device and a tubular mechanism. The tubular mechanism includes an adaptor and a tubular unit. The adaptor is removably mounted on the endoscope device. The tubular unit is removably sleeved around an insertion tube of the endoscope device through the adaptor. With the endoscope device, the adaptor and the tubular unit, the endoscope device is removably sleeved around and connected to the tubular unit. The tubular unit may serve as a catheter or a disposable envelope.

### CROSS-REFERENCE TO RELATED APPLICATION

This application claims priority to Taiwanese Patent Application No. 113124262, filed on Jun. 28, 2024, and Taiwanese Patent Application No. 113139918, filed on Oct. 21, 2024, incorporated by reference herein in their entirety.

### FIELD

The disclosure relates to imaging diagnostic equipment, and more particularly to an endoscope apparatus with an endoscope device and a tubular mechanism removably sleeved on the endoscope device.

### BACKGROUND

When inserting a nasogastric tube or a feeding tube, a conventional bendable catheter with a steel wire that is adapted to increase a supporting force of the catheter is used with an endoscope that is arranged alongside the catheter. Such arrangement of the catheter and the endoscope has a thicker structure and may cause discomfort to the patient during the insertion process, or even injury mucosa of the patient. Similarly, when removing the endoscope, an insertion tube of the endoscope may also injury the mucosa.

Moreover, after an endoscopic examination of the esophagus and the stomach of the patient using a transnasal endoscope or a transoral endoscope, the insertion tube of the endoscope which contacts the patient must be strictly cleaned and disinfected to prevent body fluids attached to the endoscope from infecting the next patient. However, it is still difficult to ensure that no bacteria, viruses and other infectious agents remain on the insertion tube and the endoscope.

Furthermore, conventional insertion catheters, such as urinary catheters, feeding tubes, etc., do not have any means to be connected to with endoscopes to cooperatively perform an insertion operation.

### SUMMARY

Therefore, an object of the disclosure is to provide an endoscope device, an adaptor and a tubular unit of an endoscope apparatus that can alleviate at least one of the drawbacks of the prior art.

According to an aspect of the disclosure, there is provided an endoscope device according to claim 1.

According to an aspect of the disclosure, there is provided an endoscope device according to claim 2.

According to an aspect of the disclosure, there is provided an endoscope device according to claim 8.

With the structure of the endoscope device, the insertion tube of the endoscope device is adapted to be sleeved by the tubular mechanism or the tubular unit and is removably connected thereto such that the tubular mechanism or the tubular unit moves synchronously with the endoscope device during the insertion operation, which prevents injury to mucosa of the patient during a catheter insertion operation, and prevents body fluids of the patient from contaminating the insertion tube.

According to an aspect of the disclosure, there is provided an adaptor according to claim 9.

With the structure of the adaptor, the tubular unit is removably sleeved around and connected to the insertion tube of the endoscope device.

According to an aspect of the disclosure, there is provided an adaptor according to claim 18.

According to an aspect of the disclosure, there is provided an adaptor according to claim 23.

According to an aspect of the disclosure, there is provided an adaptor according to claim 28.

According to an aspect of the disclosure, there is provided an adaptor according to claim 32.

With the structure of the tubular unit, the tubular unit is adapted to be mounted outwardly of the insertion tube of the endoscope device in a direct manner or through the adaptor. The drawback of the conventional endoscope device arranged alongside the catheter may be resolved, and body fluids of the patient may be prevented from contaminating the endoscope device.

### BRIEF DESCRIPTION OF THE DRAWINGS

Other features and advantages of the disclosure will become apparent in the following detailed description of the embodiments with reference to the accompanying drawings. It is noted that various features may not be drawn to scale.

FIG. 1 is a fragmentary front view illustrating a first embodiment of an endoscope apparatus according to the disclosure.

FIG. 2 is a fragmentary, exploded perspective view of the first embodiment.

FIG. 3 is a fragmentary sectional view of the first embodiment.

FIG. 4 is a fragmentary sectional view illustrating a second embodiment of the endoscope apparatus according to the disclosure.

FIG. 5 is a fragmentary, exploded perspective view illustrating a third embodiment of the endoscope apparatus according to the disclosure.

FIG. 6 is a fragmentary sectional view of the third embodiment.

FIG. 7 is a fragmentary plan view illustrating a fourth embodiment of the endoscope apparatus in a first form.

FIG. 8 is a fragmentary plan view similar to FIG. 7, illustrating the fourth embodiment in a second form.

FIG. 9 is a fragmentary sectional view illustrating an adaptor and a tubular unit of the embodiments in another form.

FIG. 10 is a fragmentary, exploded perspective view illustrating a fifth embodiment of the endoscope apparatus according to the disclosure.

FIG. 11 is a fragmentary sectional view of the fifth embodiment.

FIG. 12 is a fragmentary sectional view illustrating a sixth embodiment of the endoscope apparatus according to the disclosure.

FIG. 13 is a fragmentary sectional view illustrating the fifth embodiment and the sixth embodiment in another form.

FIG. 14 is a fragmentary, exploded perspective view illustrating a seventh embodiment of the endoscope apparatus according to the disclosure.

FIG. 15 is a fragmentary sectional view illustrating an adaptor of the seventh embodiment.

FIG. 16 is a fragmentary sectional view of the seventh embodiment.

FIG. 17 is a fragmentary, exploded perspective view illustrating an eighth embodiment of the endoscope apparatus according to the disclosure.

FIG. 18 is a cross-sectional view illustrating an adaptor of the eighth embodiment.

FIG. 19 is a fragmentary, exploded perspective view illustrating a ninth embodiment of the endoscope apparatus according to the disclosure.

FIG. 20 is a fragmentary sectional view of the ninth embodiment.

FIG. 21 is a fragmentary sectional view illustrating a tenth embodiment of the endoscope apparatus according to the disclosure.

FIG. 22 is a fragmentary sectional view illustrating a tubular unit of an eleventh embodiment of the endoscope apparatus according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

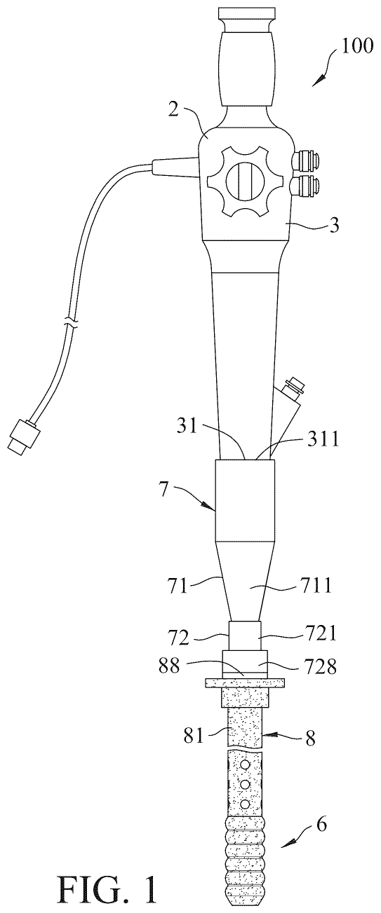

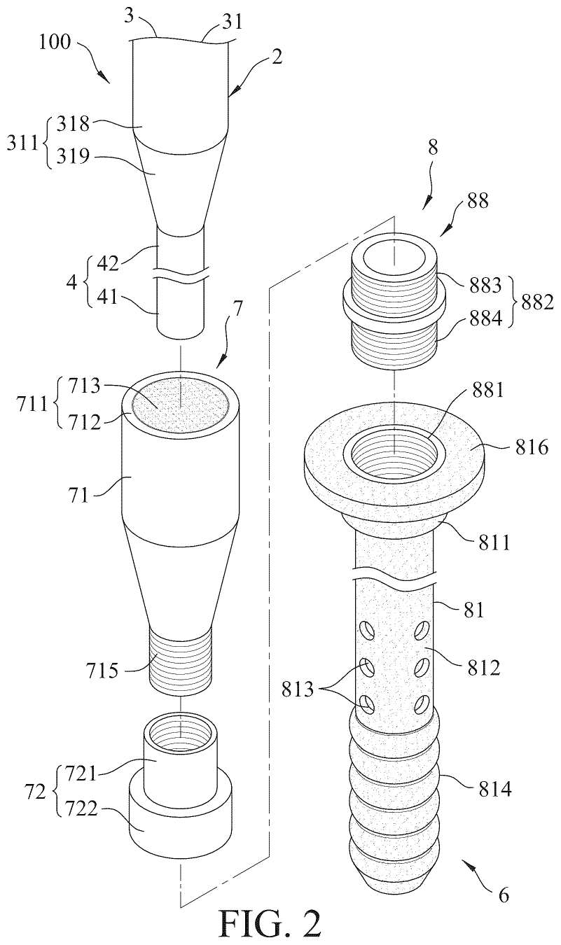

Referring to FIGS. 1 to 3, a first embodiment of an endoscope apparatus 100 according to the disclosure includes an endoscope device 2 and a tubular mechanism 6 removably mounted on the endoscope device 2.

The endoscope device 2 is sleeved by and connected to the tubular mechanism 6, and includes an endoscope unit 3, an insertion tube 4 having a proximal end connected to the endoscope unit 3 and inserted into a patient, and an image fetcher 5 disposed at a distal end of the insertion tube 4.

The endoscope unit 3 includes a frusto-conical taper head module 31 having a taper head 311. The taper head 311 has an equivalent diameter section 318 and a frusto-conical tapered section 319 gradually tapered from the equivalent diameter section 318 in an inserting direction toward the insertion tube 4.

The insertion tube 4 has a connecting section 42 connected to a narrow end of the tapered section 319, and an insertion section 41 integrally extending from the connecting section 42 to be inserted into the patient.

The image fetcher 5 is disposed at the distal end of the insertion section 41 for performing image fetching at a front of the insertion section 41 and for transmitting the images fetched to a control unit (not shown) of the endoscope unit 3. For example, the image fetcher 5 is a CCD. Since the structure of the endoscope device 2 may be of various known types, a detailed description thereof is omitted.

The tubular mechanism 6 includes an adaptor 7 which is removably mounted on the endoscope device 5, and a tubular unit 8 which is removably sleeved around the insertion tube 4 and is removably engaged with the adaptor 7.

The adaptor 7 includes an adaptor head 71 removably sleeved on the taper head 311, and an adaptor ring 72 removably connected to the adaptor head 71.

The adaptor head 71 has a proximal head section 711 configured to be removably and securely sleeved on the taper head 311, and a distal head section 715 extending co-axially from a narrow end of the proximal head section 711 to be sleeved on the connecting section 42 of the insertion tube 4. The proximal head section 711 has a surrounding wall 712 connected to the distal head section 715 to be sleeved on the taper head 311, and an adhesive layer 713 disposed on an inner surface of the surrounding wall 712 to be removably attached to an outer peripheral surface of the taper head 311. The distal head section 715 has external threads formed on an outer peripheral surface thereof to be threadedly engaged with the adaptor ring 72. The adhesive layer 713 may be in the form of a reusable adhesive tape, such as an acrylic adhesive tape, which is to be removably attached to the outer peripheral surface of the taper head 311.

The adaptor ring 72 has a first ring section 721 which is removably and threadedly engaged with the distal head section 715 of the adaptor head 71, and a second ring section 722 which surrounds and is spaced apart from the connecting section 42 of the insertion tube 4 to be removably engaged with the tubular unit 8. The second ring section 722 has a diameter larger than that of the first ring section 721, and has internal threads formed on an inner peripheral surface thereof.

The tubular unit 8 is in the form of a feeding tube in this embodiment. The tubular unit 8 includes a flexible tubular body 81 removably sleeved around the insertion tube 4, and a connecting assembly 88 securely and coaxially mounted on the flexible tubular body 81 to be removably engaged with the adaptor ring 72 of the adaptor 7.

The flexible tubular body 81 is made of a flexibly deformable material, such as silicone rubber or latex, and has a proximal end portion 811, an extension portion 812 extending axially from the proximal end portion 811, and a distal end portion 814 in the form of a worm tube extending axially from the extension portion 812. A flange 816 is formed on and extends radially from the proximal end portion 811. The extension portion 812 has a plurality of radial holes 813 formed adjacent to the distal end portion 814 and radially extending therethrough. The distal end portion 814 has an end opening 815.

The connecting assembly 88 includes a fixed sleeve 881 which is coaxially and securely mounted on the proximal end portion 811 of the flexible tubular body 81 and has internal threads, and a connecting sleeve 882 which is removably engaged with the fixed sleeve 881 and the second ring section 722 of the adaptor ring 72 to be removably engaged with the adaptor 7. Specifically, the connecting sleeve 882 has a first engaging section 883 inserted into and threadedly engaged with the second ring section 722 and sleeved around the connecting section 42, and a second engaging section 884 inserted into and threadedly engaged with the fixed sleeve 881.

When the tubular unit 8 serving as a feeding tube is used with the adaptor 7 and the endoscope device 2 to perform an insertion operation, the insertion tube 4 extends through the adaptor head 71, the adaptor ring 72, the connecting sleeve 882 and the fixed sleeve 881 and into the flexible tubular body 81. The adaptor ring 72 is sequentially connected to the connecting sleeve 882 and the adaptor head 71, and then the connecting sleeve 882 is connected to the fixed sleeve 881. Next, the proximal head section 711 of the adaptor head 71 is securely sleeved on the taper head 311 to assemble the endoscope device 2, the adaptor 7 and the tubular unit 8 together.

During the insertion operation, the tubular unit 8 moves synchronously with the endoscope device 2 and the adaptor 7. With the image fetcher 5 of the endoscope device 2 mounted on the distal end of the insertion tube 4, images in front of the tubular body 81 in the inserting direction are fetched by the image fetcher 5 through the end opening 815. Furthermore, by virtue of the distal end portion 814 in the form of a worm tube, the distal end portion 814 of the tubular body 81 is flexibly deformed by the insertion tube 4 of the endoscope device 2. In other embodiments, the distal end portion 814 may be in another flexibly deformable form.

Since the tubular body 81 is sleeved around the insertion tube 4, the drawback of the conventional endoscope device arranged alongside the catheter may be resolved. When it is desired to remove the insertion tube 4 from the tubular unit 8 after the insertion operation of the tubular unit 4 is completed, the insertion tube 4 is smoothly moved away from the tubular unit 8 and does not hurt the patient.

In this embodiment, the tubular body 81 serves as a feeding tube such that the radial holes 813 are formed therein to provide fluid food from the radial holes 813. The tubular body 81 may serve as a urinary catheter. In this case, the tubular body 81 is free of radial holes 813, and the distal end portion 814 is not in the form of a worm tube.

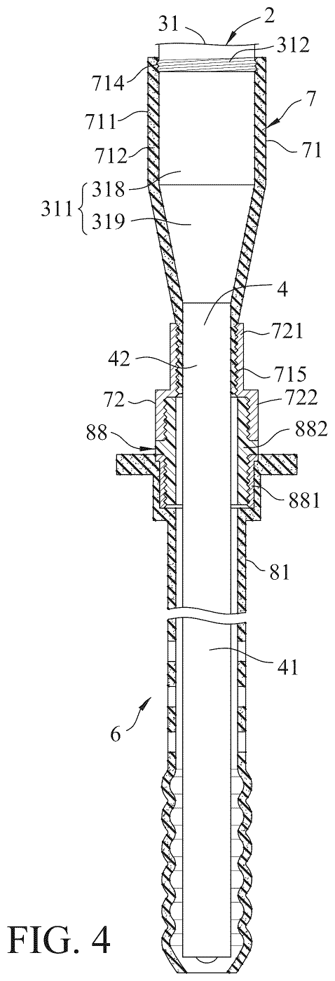

With reference to FIGS. 1 and 4, in a second embodiment of the disclosure, an outer peripheral surface of the equivalent diameter section 318 of the taper head 311 of the endoscope unit 3 is formed with a first connecting feature 312. An inner peripheral surface of the surrounding wall 712 of the adaptor head 71 is formed with a second connecting feature 714. The first connecting feature 312 and the second connecting feature 714 are respectively in the form of external threads and internal threads which are to be removably and threadedly engaged with each other. Thus, the surrounding wall 712 of the adaptor head 71 is removably and securely sleeved on the taper head 311.

In other embodiments, the first connecting feature 312 and the second connecting feature 714 may be in the form of male and female engaging structures.

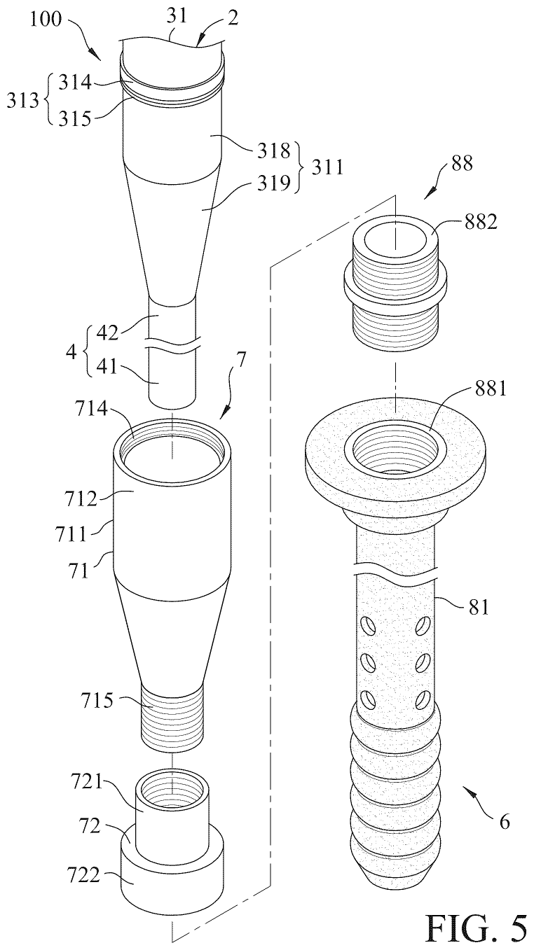

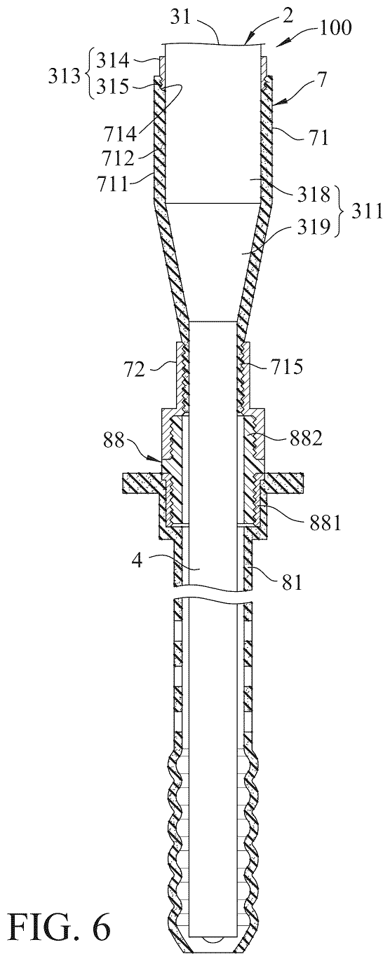

With reference to FIGS. 5 and 6, in a third embodiment, the taper head module 31 includes a taper head 311 and a connecting ring 313 which is sleeved on an outer peripheral surface of the equivalent diameter section 318 of the taper head 311. The connecting ring 313 has a ring body 314 which is securely sleeved on the outer peripheral surface of the taper head 311, and a first connecting feature 315 which is formed on an outer peripheral surface of the ring body 314 to be removably engaged with the second connecting feature 714 of the adaptor head 71. The first connecting feature 315 and the second connecting feature 714 are respectively in the form of external threads and internal threads which are to be removably and threadedly engaged with each other. Thus, the adaptor head 71 of the tubular mechanism 6 is removably and securely sleeved on the taper head module 31 by a threaded engagement of the second connecting feature 714 with the first connecting feature 315.

Alternatively, the connecting ring 313 may be integrally formed and connected to the taper head 311.

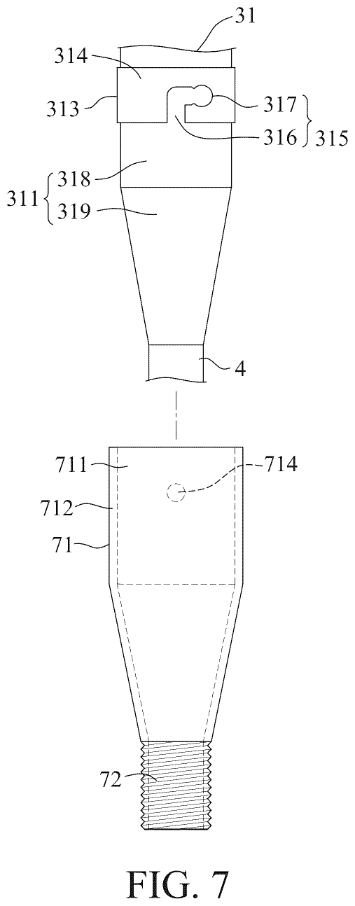

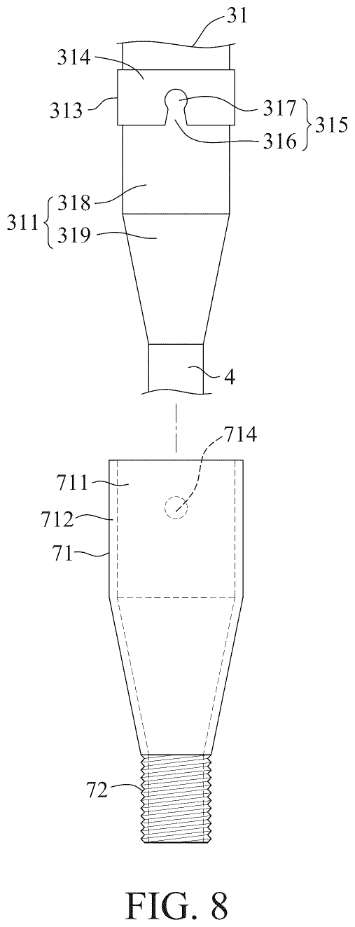

With reference to FIGS. 7 and 8, in a fourth embodiment, the taper head module 31 includes a taper head 311 and a connecting ring 313. The connecting ring 313 has two first connecting features 315 formed on an outer peripheral surface thereof and diametrically opposite to each other (only one is shown in the drawings). The adaptor head 71 has two second connecting features 714 formed on an inner peripheral surface thereof and diametrically opposite to each other (only one is shown in the drawings).

Each first connecting feature 315 and each second connecting feature 714 are in the form of snap fasteners which are removably engaged with each other. For example, the first connecting feature 315 is an engaging slot, and the second connecting feature 714 is an engaging stud, and vice versa. Referring to FIG. 7, the first connecting feature 315 is an L-shaped engaging slot formed in the outer peripheral surface of the connecting ring 313, and has an insertion section 316 extending in the inserting direction and opened toward the insertion tube 4, and an engaging section 317 extending transversely from the axially extending section 316. The second connecting feature 714 is in the form of the cylindrical stud projecting from the inner peripheral surface of the proximal head section 711 of the adaptor head 71. To connect the adaptor head 71 to the endoscope device 2, the adaptor head 71 is moved toward the endoscope unit 3 to engage the second connecting features 714 with the insertion sections 316, and the adaptor head 71 is then rotated relative to the taper head module 31 to engage the second connecting features 714 with the engaging sections 317.

Referring to FIG. 8, in another form, the first connecting feature 315 is an axially extending slot formed in the outer peripheral surface of the connecting ring 313 and having an insertion section 316 and an engaging section 317 which extends along the insertion direction from the insertion section 316. To connect the adaptor head 71 to the endoscope device 2, the adaptor head 71 is moved toward the endoscope unit 3 to engage the second connecting features 714 with the insertion sections 316, and the adaptor head 71 is then pressed and further moved to engage the second connecting features 714 with the engaging sections 317.

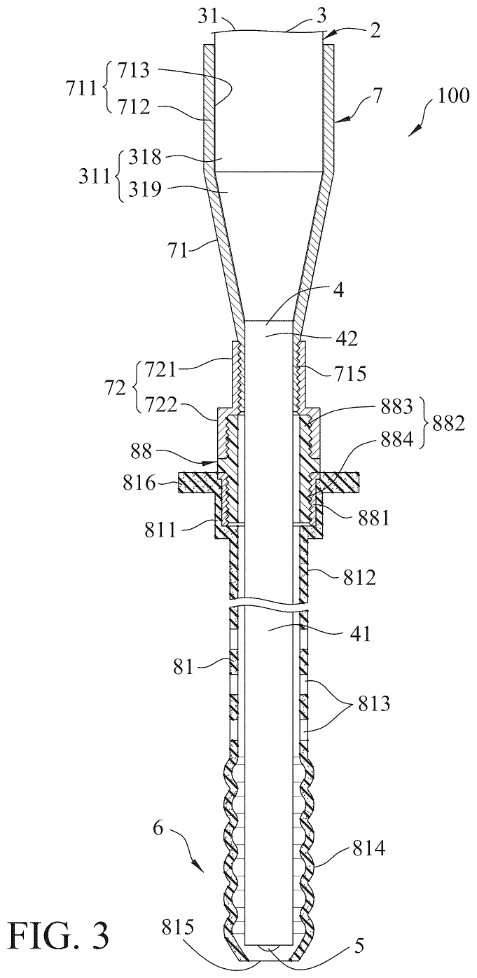

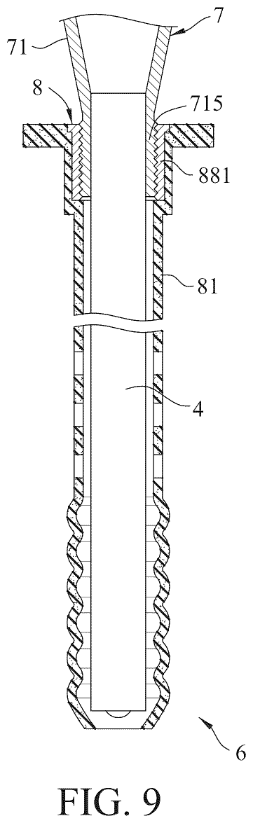

With reference to FIG. 9, alternatively, in the previous embodiments, the adaptor ring 72 (as shown in FIGS. 3, 4 and 6) of the adaptor 7 may be dispensed with, and the connecting sleeve 882 (as shown in FIGS. 3, 4 and 6) of the connecting assembly 88 may be dispensed with. Specifically, the fixed sleeve 881 is directly and threadedly engaged with the distal head section 715 of the adaptor head 71 so as to removably and securely sleeve the tubular unit 8 on the insertion tube 4.

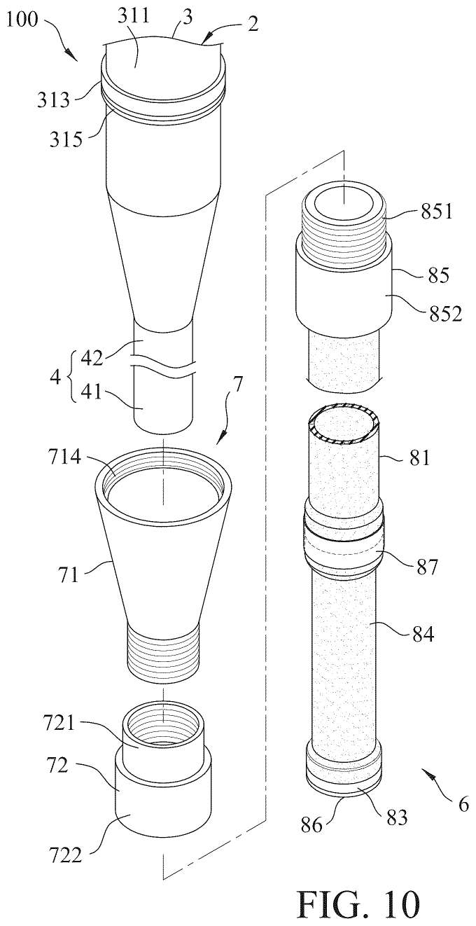

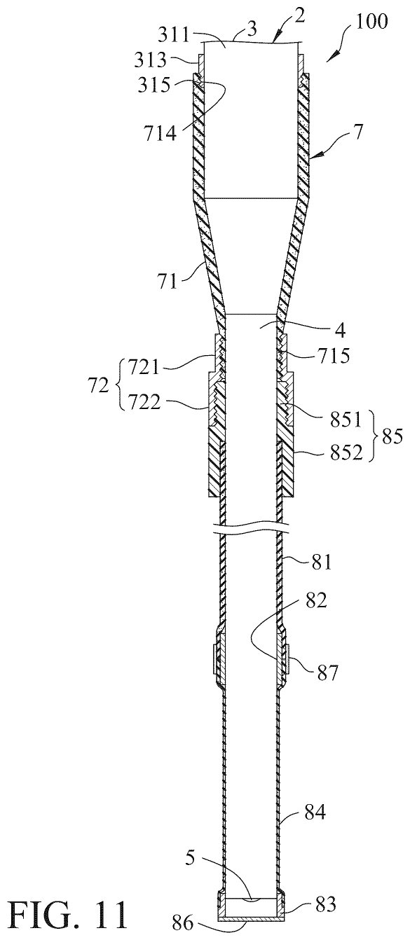

With reference to FIGS. 10 and 11, in a fifth embodiment, the tubular unit 8 includes an engaging ring 82 and an end ring 83 which are axially spaced apart from each other, a flexible terminal tube 84 which has two ends respectively sleeved on and communicated with the engaging ring 82 and the end ring 83, a flexible tubular body 81 which has a distal end sleeved on and communicated with the engaging ring 82, an end cap 83 which is securely disposed on the end ring 83 to cover an opening of the end ring 83 and which is made of a translucent material, a wrap tape 87 which surrounds and wraps the distal end of the flexible tubular body 81 and the end of the flexible terminal tube 84 to cover and seal a junction between the flexible tubular body 81 and the flexible terminal tube 84, and an adaptor tube 85 which has a first tube section 851 removably and threadedly engaged with the second ring section 722 of the adaptor ring 72, and a second tube section 852 securely and coaxially engaged with a proximal end of the flexible tubular body 81 distal from the engaging ring 82.

Specifically, the distal end of the flexible tubular body 81 is connected to the end of the flexible terminal tube 84. Each of the flexible tubular body 81 and the flexible terminal tube 84 may be made of a flexibly deformable material which is to be deformed radially and tensible axially. Alternatively, the flexible terminal tube 84 may be made of an elastomeric material which is to be bendably deformed. A wall thickness of the flexible terminal tube 84 is smaller than that of the flexible tubular body 81. For example, the flexible tubular body 81 and the flexible terminal tube 84 may be made of polyisoprene, silicone latex, silicone rubber, PU (polyurethane), TPE (thermoplastic elastomer), etc. In this embodiment, the tubular body 81 and the terminal tube 84 are made of different materials. For example, the tubular body 81 may be made of silicone rubber or latex, and the terminal tube 84 may be made of PU or polyisoprene.

To mount the tubular unit 8 outwardly of an insertion tube 4 of an endoscope device 2 through the adaptor 7, the adaptor ring 72 is securely sleeved on the adaptor tube 85, and is then sleeved around the insertion tube 4 from the insertion section 41 toward the connecting section 42. During the sleeving movement, the flexible tubular body 81 is flexibly enlarged by the insertion section 41 and is fittingly sleeved on the insertion section 41 to permit the insertion section 41 to insert into the flexible terminal tube 84. Subsequently, the adaptor ring 72 is sleeved on the distal head section 715 of the adaptor head 71.

In this embodiment, the tubular unit 8 serves as a disposable envelope to prevent body fluids from contaminating the insertion tube 4 during the insertion and inspection of the endoscope device 2. Through the translucent end cap 86, the image fetcher 5 performs the image fetching.

When it is desired to remove the endoscope device 2 and the tubular unit 8 serving as a disposable envelope from the patient after the operation of the endoscope device 2 is completed, the adaptor tube 85 is removed from the adaptor ring 72, and the entire tubular unit 8 is then removed from the endoscope device 2 for disposal. Since the endoscope device 2 is not contaminated with the patient's body fluids, complexity of disinfecting the endoscope device 2 is greatly reduced.

In practice, the wall diameter of the flexible tubular body 81 and the flexible terminal tube 84 is designed to be flexibly and fittingly sleeved on the insertion tube 4 of the endoscope device 2. In other embodiments, the end cap 86 of the tubular unit 8 may be dispensed with.

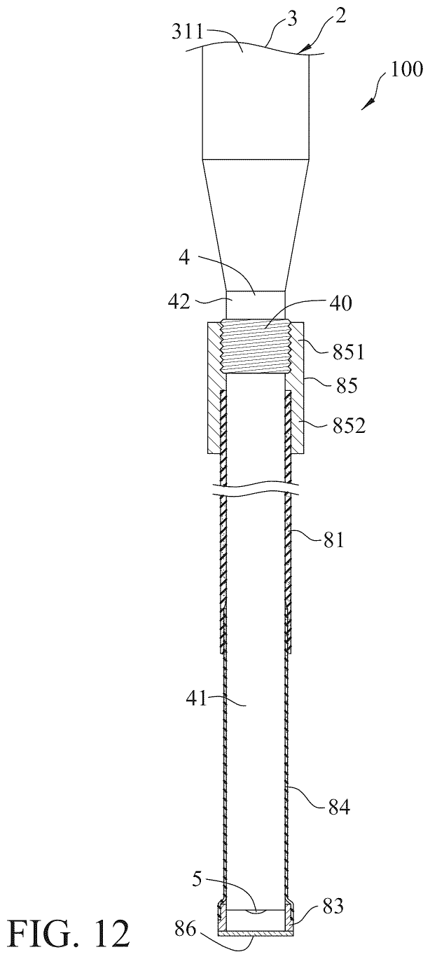

With reference to FIG. 12, in a sixth embodiment of the endoscope apparatus 100, the adaptor is dispensed with. Specifically, the connecting section 42 of the insertion tube 4 has an external thread structure 40 formed on an outer peripheral surface thereof. The first tube section 851 of the adaptor tube 85 has internal threads to be removably and threadedly engaged with the external thread structure 40. Additionally, the engaging ring 82 of the tubular unit 8 (see FIG. 11) is dispensed with. The flexible tubular body 81 is securely connected to and communicated with the terminal tube 84. For example, the distal end of the tubular body 81 is sleeved on and connected to the end of the terminal tube 84 and is secured to the terminal tube 84 by adhesives or sewing threads.

To mount the tubular unit 8 on the endoscope device 2, the flexible tubular body 81 and the flexible terminal tube 84 are sleeved around the insertion tube 4, and the adaptor tube 85 is then threadedly engaged with the external thread structure 40 of the insertion tube 4.

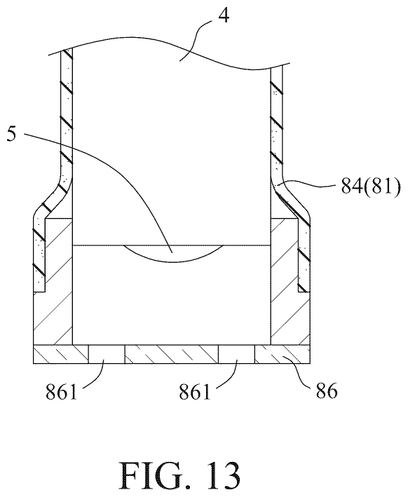

With reference to FIG. 13, in the previous embodiments, the end cap 86 has apertures 861 extending therethrough for passing of parts (not shown) of the endoscope device 2 or for allowing the endoscope device 2 to perform an inflation procedure or a suction procedure.

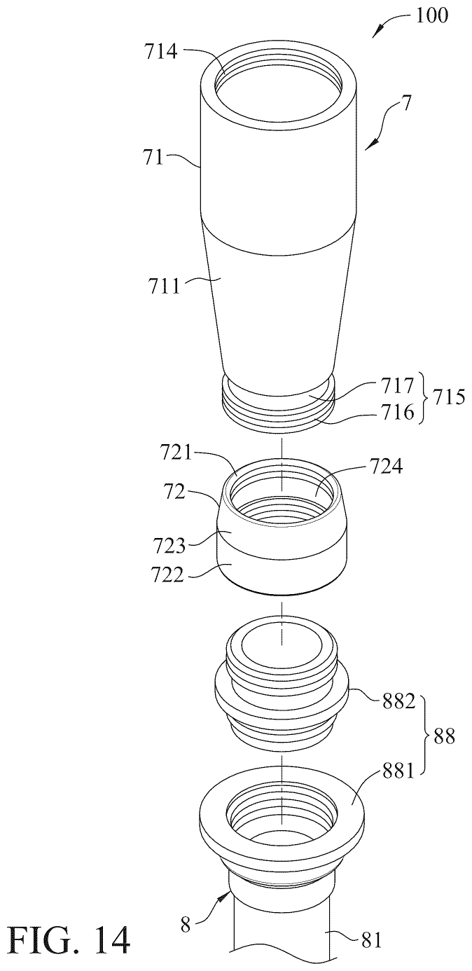

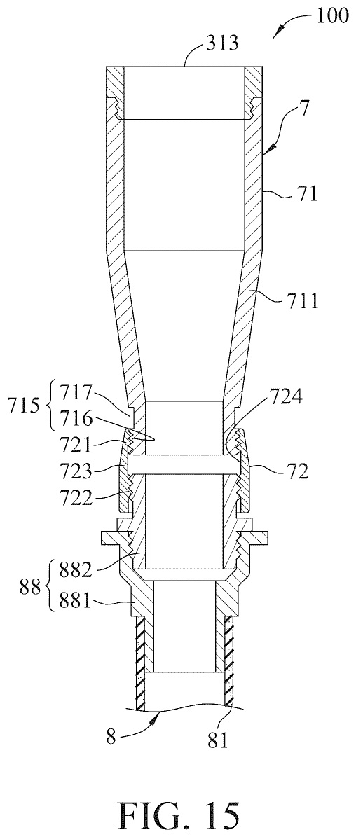

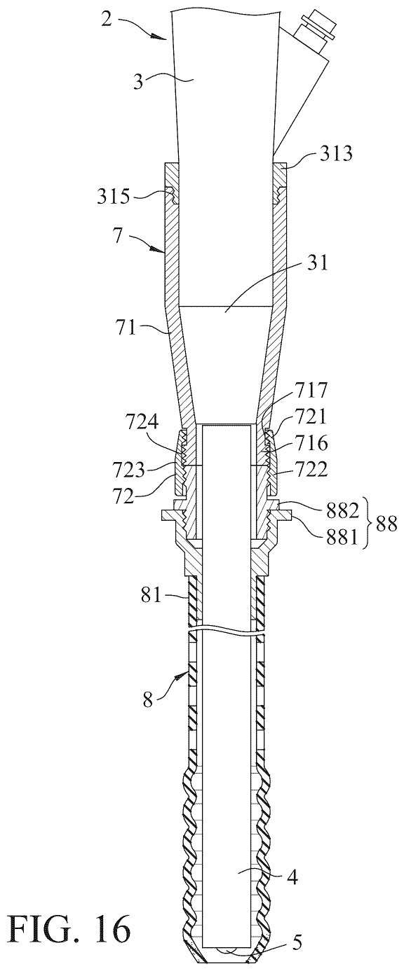

With reference to FIGS. 14, 15 and 16, in a seventh embodiment, the adaptor 7 has a structure different from that of the third embodiment.

The distal head section 715 of the adaptor head 71 has an externally threaded portion 716 formed on an outer peripheral surface thereof, and an outer annular groove 717 formed in the outer peripheral surface and interposed between the externally threaded portion 716 and the proximal head section 711. The adaptor ring 72 further has a transition section 723 which is interposed between the first ring section 721 and the second ring section 722 and which defines an inner annular groove 724.

The adaptor ring 72 is axially movable relative to the distal head section 715 so as to be shiftable between a threadedly engaging position and an idling position. In the threadedly engaging position, the first ring section 721 of the adaptor ring 72 is threadedly engaged with the externally threaded portion 716. In the idling position, the first ring section 721 is rotatably engaged in the outer annular groove 717 and the externally threaded portion 716 is rotatably engaged in the inner annular groove 724.

To connect the adaptor 7 to the tubular unit 8, the first ring section 721 of the adaptor ring 72 is threadedly engaged with the externally threaded portion 716 of the distal head section 715 to move the first ring section 721 toward the proximal head section 711, until the internal threads of the first ring section 721 are engaged in the outer annular groove 717 while the externally threaded portion 716 is engaged in the inner annular groove 724. At this time, the adaptor ring 72 is freely rotatable relative to the adaptor head 71. Subsequently, the adaptor ring 72 is rotated relative to the adaptor head 71 to bring the second ring section 722 in threaded engagement with the connecting sleeve 882 of the tubular unit 8. Next, the insertion tube 4 of the endo scope device 2 is inserted into the tubular body 81 through the adaptor 7. The adaptor head 71 is rotated relative to the adaptor ring 72 to bring the adaptor head 71 in threaded engagement with the connecting ring 313.

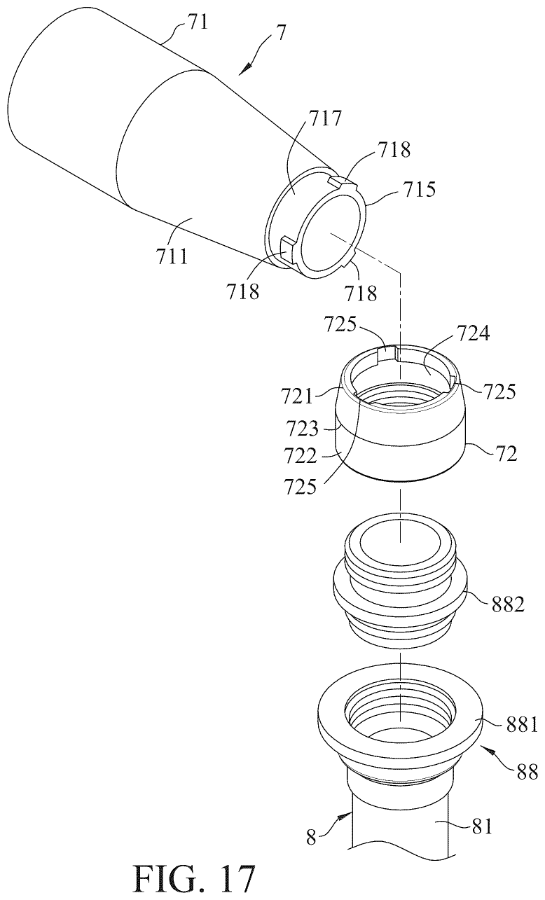

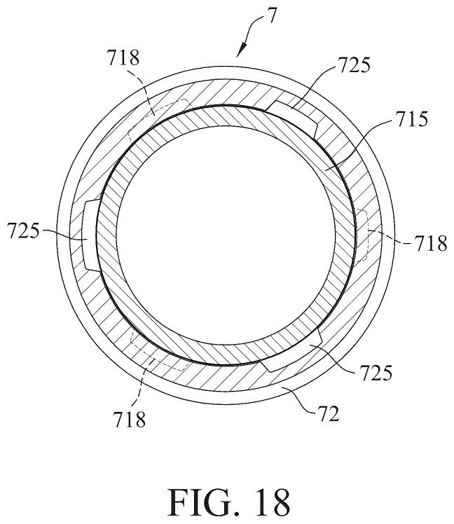

With reference to FIGS. 17 and 18, in an eighth embodiment, the adaptor 7 has a structure different from that of the seventh embodiment.

The distal head section 715 of the adaptor head 71 has three aligning protrusions 718 which protrude from an outer peripheral surface thereof and which are angularly spaced apart from each other, and an outer annular groove 717 which is defined by the outer peripheral surface and which is interposed between the aligning protrusions 718 and the proximal head section 711. The aligning protrusions 718 respectively have different widths along a periphery of the outer peripheral surface.

The adaptor ring 72 has a transition section 723 which is interposed between the first ring section 721 and the second ring section 722 and which defines an inner annular groove 724. The first ring section 721 protrudes radially and inwardly relative to the transition section 723 and has a plurality of aligning notches 725 which extend axially to communicate with the inner annular groove 724 and which respectively have different widths along a periphery thereof.

In this embodiment, the widths of the aligning protrusions 718 respectively correspond with the widths of the aligning notches 725 to be matingly engaged with and axially aligned with the aligning notches 725, respectively.

To connect the adaptor ring 72 to the adaptor head 71, the aligning notches 725 of the adaptor ring 72 are respectively aligned with the corresponding aligning protrusions 718, and the adaptor ring 72 is moved toward the adaptor head 71 to bring the aligning protrusions 718 through the aligning notches 725 and into the inner annular groove 724. The adaptor ring 72 is then rotated relative to the adaptor head 71 to engage the first ring section 721 in the outer annular groove 717 and engage the aligning protrusions 718 in the inner annular groove 724 such that the adaptor ring 72 is freely rotatable relative to the adaptor head 71.

In this embodiment, three of the aligning notches 725 are formed in the adaptor ring 72, and three of the aligning protrusions 718 are formed on the adaptor head 71. Alternatively, the number of the aligning notches 725 and the aligning protrusions 718 may be varied.

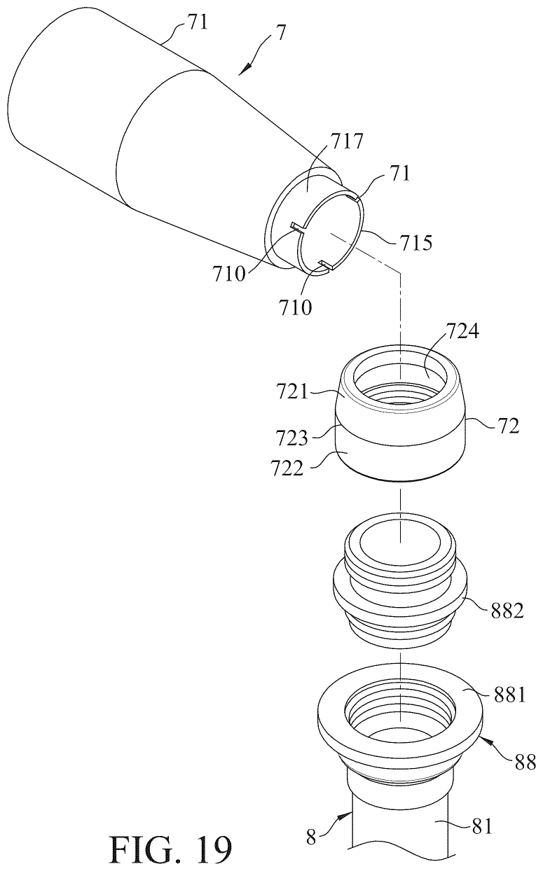

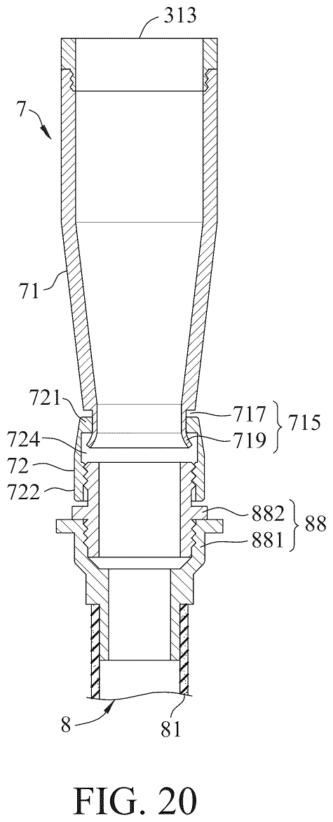

With reference to FIGS. 19 and 20, in a ninth embodiment, the adaptor 7 has a structure different from that of the seventh embodiment.

The distal head section 715 of the adaptor head 71 has a plurality of splits 710 formed therethough and angularly spaced apart from each other, and a plurality of retaining protrusions 719 formed on an outer peripheral surface thereof and each interposed between two adjacent splits 710. Each retaining protrusion 719 extends and is bent radially from an end wall of the distal head section 715 to be rotatably engaged in the inner annular groove 724 of the adaptor ring 72. The distal head section 715 further has an outer annular groove 717 which is defined by the outer peripheral surface and which is interposed between the retaining protrusions 718 and the proximal head section 711.

The first ring section 721 of the adaptor ring 72 protrudes radially and inwardly relative to the transition section 723 and is free of internal threads to be rotatably engaged in the outer annular groove 717.

In this embodiment, three of the splits 710 are formed in the distal head section 715 of the adaptor head 71, and three of the retaining protrusions 719 are formed on the distal head section 715 of the adaptor head 71. Alternatively, the number of the splits 710 and the retaining protrusions 719 may be varied, such as two.

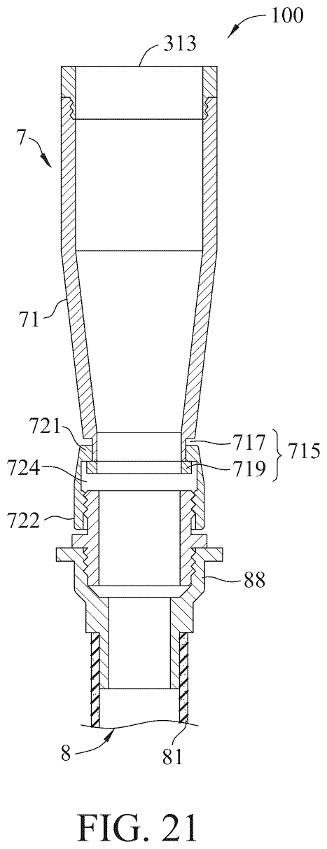

With reference to FIG. 21, in a tenth embodiment, the adaptor 7 has a structure different from that of the seventh embodiment.

The distal head section 715 of the adaptor head 71 has an outer annular groove 717 formed in an outer peripheral surface thereof. The first ring section 721 of the adaptor ring 72 protrudes radially and inwardly relative to the transition section 723 and is free of internal threads to be rotatably engaged in the outer annular groove 717.

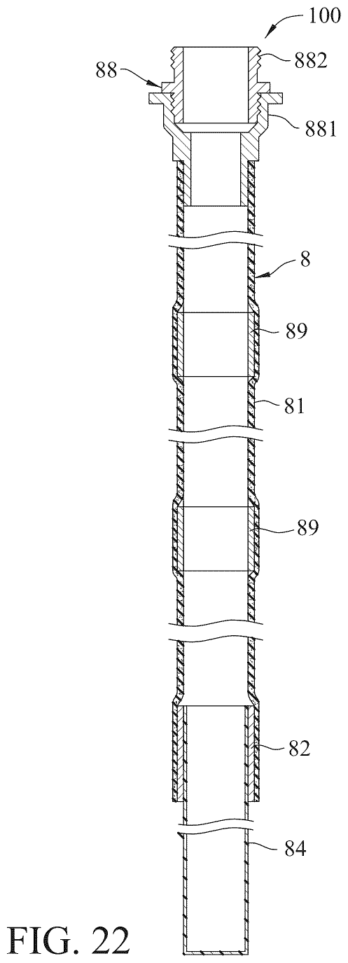

With reference to FIG. 22, in an eleventh embodiment, the tubular unit 8 has a structure different from that of the fifth embodiment.

The tubular unit 8 includes a connecting assembly 88 and an engaging ring 82 which are axially spaced apart from each other, a flexible tubular body 81 which has proximal and distal ends sleeved on and communicated with the connecting assembly 88 and the engaging ring 82 and which is flexibly deformable and tensible axially, a plurality of support rings 89 which are disposed within and communicated with the flexible tubular body 81 and which are spaced apart from each other along a lengthwise direction of the flexible tubular body 81, and a flexible terminal tube 84 which has a first end securely engaged and communicated with the distal end of the flexible tubular body 81 through the engaging ring 82. The flexible terminal tube 84 extends outwardly of the engaging ring 82 to terminate at a second end. Each of the flexible tubular body 81 and the flexible terminal tube 84 is made of a translucent material.

The connecting assembly 88 has a fixed sleeve 881 which is coaxially and securely mounted on the proximal end of the flexible tubular body 81, and a connecting sleeve 882 which is removably engaged with the fixed sleeve 881. With the support rings 89 disposed within the flexible tubular body 81, the insertion tube 4 of the endoscope device 2 may be inserted into the tubular unit 8 more easily. The number of the support rings 89 may be varied as required.

As illustrated, with the structure of the endoscope device 2 and the structures of the adaptor 7 and the tubular unit 8 of the tubular mechanism 6, the tubular unit 8 is adapted to be mounted outwardly of the insertion tube 4 of the endoscope device 2 in a direct manner or through the adaptor 7.

When the tubular unit 8 serves as a feeding tube or a urinary catheter, the insertion tube 4 of the endoscope device 2 is inserted into the tubular unit 8 to perform a catheter insertion operation. The image fetcher 5 is disposed to perform image fetching at the front of the insertion tube 4 through the end opening 815 of the tubular body 81. When the tubular unit 8 serves as a disposable envelope, body fluids may be prevented from contaminating the insertion tube 4 during the insertion and inspection of the endoscope device 2 so as to reduce the complexity of the disinfecting of the endoscope device 2.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. An endoscope device adapted to be sleeved by and connected to a tubular mechanism, comprising:
an insertion tube insertable and mountable into the tubular mechanism in an inserting direction, and having a proximal end and a distal end; and
an endoscope unit connected to said proximal end of said insertion tube, and having a taper head which is gradually tapered in the inserting direction, said taper head having an outer peripheral surface which is formed with a first connecting feature to be removably and threadedly engaged with the tubular mechanism sleeved around said insertion tube.

2. An endoscope device adapted to be sleeved by and connected with a tubular mechanism, comprising:
an insertion tube insertable and mountable into the tubular mechanism in an inserting direction, and having a proximal end and a distal end; and
an endoscope unit connected with said proximal end of said insertion tube, and having a taper head which is gradually tapered in the inserting direction, and a connecting ring which is sleeved on an outer peripheral surface of said taper head, said connecting ring having a ring body which is securely sleeved on said outer peripheral surface of said taper head, and a first connecting feature which is formed on an outer peripheral surface of said ring body to be removably engaged with the tubular mechanism sleeved around said insertion tube.

3. The endoscope device of claim 2, wherein said first connecting feature is in form of external threads to be removably and threadedly engaged with the tubular mechanism sleeved around said insertion tube.

4. The endoscope device of claim 2, wherein said first connecting feature is in form of a snap fastener to be removably engaged with the tubular mechanism sleeved around said insertion tube.

5. The endoscope device of claim 4, the tubular mechanism having a second connecting feature in form of a snap fastener, wherein said first connecting feature is in form of an engaging slot which is formed in said outer peripheral surface of said ring body, said first connecting feature having an insertion section which is opened in the insertion direction, and an engaging section which extends transverse to said insertion section.

6. The endoscope device of claim 4, the tubular mechanism having a second connecting feature in form of a snap fastener, wherein said first connecting feature is in form of an engaging slot which is formed in said outer peripheral surface of said ring body, said first connecting feature having an insertion section which is opened in the insertion direction, and an engaging section which extends along the insertion direction from said insertion section.

7. The endoscope device of claim 2, wherein said connecting ring is integrally formed with and connected to said taper head.

8. An endoscope device adapted to be sleeved by and connected to a tubular unit, comprising:
an endoscope unit; and
an insertion tube having a proximal end which is connected to said endoscope unit, and insertable and mountable into the tubular unit, said insertion tube having an outer peripheral surface which faces the tubular unit and which has a threaded structure to be removably and threadedly engaged with the tubular unit sleeved around said insertion tube.

9. An adaptor for removably connecting a tubular unit with an endoscope device, the endoscope device including an endoscope unit and an insertion tube which is connected with the endoscope unit and is insertable and mountable into the tubular unit in an inserting direction, the endoscope unit having a taper head which is gradually tapered in the inserting direction, said adaptor comprising:
a adaptor head having a proximal head section to be removably and securely sleeved on the taper head, and a distal head section to be sleeved on the insertion tube; and
an adaptor ring sleeved around the tubular unit, and having a first ring section which is coaxially connected to said distal head section, and a second ring section which is removably engaged with the tubular unit.

10. The adaptor of claim 9, wherein said proximal head section has a surrounding wall to be sleeved on the taper head, and an adhesive layer disposed on an inner surface of said surrounding wall to be removably attached to an outer peripheral surface of the taper head.

11. The adaptor of claim 9, the taper head having a first connecting feature formed on an outer peripheral surface thereof and in form of external threads, wherein said proximal head section has a second connecting feature to be removably and threadedly engaged with the first connecting feature.

12. The adaptor of claim 9, the taper head having two first connecting features formed on an outer peripheral surface thereof and in form of engaging slots, wherein said proximal head section has a surrounding wall to be sleeved on the taper head, and two second connecting features in form of snap fasteners which project from an inner surface of said surrounding wall to be respectively and removably engaged in the first connecting features.

13. The adaptor of claim 9, wherein said distal head section of said adaptor head has an externally threaded portion formed on an outer peripheral surface thereof, said first ring section of said adaptor ring being sleeved around said distal head section and being threadedly engaged with said externally threaded portion, said second ring section of said adaptor ring being configured for threadedly engaging with the tubular unit.

14. The adaptor of claim 9, wherein said first ring section of said adaptor ring is rotatably sleeved on said distal head section.

15. The adaptor of claim 14, wherein said distal head section has an externally threaded portion formed on an outer peripheral surface thereof, and an outer annular groove formed in said outer peripheral surface and interposed between said externally threaded portion and said proximal head section, said adaptor ring further having a transition section which is interposed between said first ring section and said second ring section and which defines an inner annular groove such that said adaptor ring is movable axially relative to said distal head section to be shiftable between a threadedly engaging position, where said first ring section of said adaptor ring is threadedly engaged with said externally threaded portion, and an idling position, where said first ring section is rotatably engaged in said outer annular groove and said externally threaded portion is rotatably engaged in said inner annular groove.

16. The adaptor of claim 14, wherein said adaptor ring has a transition section which is interposed between said first ring section and said second ring section and which defines an inner annular groove, said first ring section protruding radially and inwardly relative to said transition section and having a plurality of aligning notches which extend axially to communicate with said inner annular groove and which respectively have different widths along a periphery thereof, said distal head section of said adaptor head having a plurality of aligning protrusions which protrude from an outer peripheral surface thereof and which are angularly spaced apart from each other to be rotatably engaged in said inner annular groove, and an outer annular groove defined by said outer peripheral surface and interposed between said aligning protrusions and said proximal head section such that said first ring section is rotatably engaged in said outer annular groove, said aligning protrusions respectively having different widths along a periphery of said outer peripheral surface which correspond with said widths of said aligning notches to be matingly engaged with and axially aligned with said aligning notches, respectively.

17. The adaptor of claim 14, wherein said distal head section of said adaptor head has a plurality of retaining protrusions formed on an outer peripheral surface thereof, said adaptor ring further having a transition section which is interposed between said first ring section and said second ring section and which defines an inner annular groove, said first ring section protruding radially and inwardly relative to said transition section to be rotatably engaged in said outer annular groove, said retaining protrusions being rotatably engaged in said inner annular groove.

18. A tubular unit adapted to be mounted outwardly of an insertion tube of an endoscope device in a direct manner or through an adaptor as claimed in claim 9, comprising:
a flexible tubular body removably sleeved around the insertion tube; and
a connecting assembly securely and coaxially mounted on said flexible tubular body to be removably engaged with the insertion tube or removably engaged with the adaptor.

19. The tubular unit of claim 18, wherein said connecting assembly includes a fixed sleeve which is securely mounted on said flexible tubular body to be removably engaged with the insertion tube or the adaptor.

20. The tubular unit of claim 18, wherein said connecting assembly includes a fixed sleeve which is securely mounted on said flexible tubular body, and a connecting sleeve which is removably engaged with said fixed sleeve to be removably engaged with the adaptor.

21. The tubular unit of claim 19, wherein said flexible tubular body has a proximal end portion, an extension portion extending from said proximal end portion, and a distal end portion in form of a worm tube extending from said extension portion, said fixed sleeve being coaxially engaged and communicated with said proximal end portion.

22. The tubular unit of claim 19, wherein said flexible tubular body has a proximal end portion, an extension portion extending from said proximal end portion, and a distal end portion extending from said extension portion, said extension portion having a plurality of radial holes radially extending therethrough, said fixed sleeve being coaxially engaged and communicated with said proximal end portion.

23. A tubular unit adapted to be mounted outwardly of an insertion tube of an endoscope device in a direct manner or through an adaptor as claimed in claim 9, comprising:
an engaging ring and an end ring axially spaced apart from each other;
a flexible tubular body having a distal end which is engaged and communicated with said engaging ring;
a flexible terminal tube having two ends which are respectively engaged and communicated with said engaging ring and said end ring; and
an adaptor tube having a first tube section which is removably engaged with the insertion tube or the adaptor, and a second tube section which is securely and coaxially engaged with a proximal end of said flexible tubular body distal from said engaging ring.

24. The tubular unit of claim 23, wherein said distal end of said flexible tubular body and one of said ends of said flexible terminal tube are sleeved on said engaging ring and disposed adjacent to each other, said tubular unit further comprising a wrap tape which surrounds and wraps said distal end of said flexible tubular body and said end of said flexible terminal tube to cover and seal the junction between said flexible tubular body and said flexible terminal tube.

25. The tubular unit of claim 23, further comprising an end cap which is disposed on said end ring to cover an opening of said end ring and which is made of a translucent material.

26. The tubular unit of claim 25, wherein said end cap has at least one aperture extending therethrough.

27. The tubular unit of claim 23, wherein said flexible tubular body is made of a material different from a material of said flexible terminal tube.

28. A tubular unit adapted to be mounted outwardly of an insertion tube of an endoscope device in a direct manner or through an adaptor as claimed in claim 9, comprising:
a flexible tubular body having a proximal end and a distal end;
a flexible terminal tube having a first end which is securely engaged and communicated with said distal end of said flexible tubular body;
an end ring coaxially disposed on a second end of said flexible terminal tube distal from said flexible tubular body; and
an adaptor tube having a first tube section which is removably engaged with the insertion tube or the adaptor, and a second tube section which is securely and coaxially engaged with said proximal end of said flexible tubular body distal from said flexible terminal tube.

29. The tubular unit of claim 28, further comprising an end cap which is disposed on said end ring to cover an opening of said end ring and which is made of a translucent material.

30. The tubular unit of claim 29, wherein said end cap has at least one aperture extending therethrough.

31. The tubular unit of claim 28, wherein said flexible tubular body is made of a material different from a material of said flexible terminal tube.

32. A tubular unit adapted to be mounted outwardly of an insertion tube of an endoscope device in a direct manner or through an adaptor as claimed in claim 9, comprising:
a connecting assembly to be removably engaged with the insertion tube or removably engaged with the adaptor;
an engaging ring axially spaced apart from said connecting assembly;
a flexible tubular body having a proximal end which is coaxially engaged and communicated with said connecting assembly, and a distal end which is coaxially engaged and communicated with said engaging ring to be sleeved around the insertion tube; and
a flexible terminal tube having a first end which is engaged and communicated with said engaging ring and extending outwardly from said engaging ring to be sleeved around the insertion tube.

33. The tubular unit of claim 32, further comprising at least one support ring which is disposed within and communicated with said flexible tubular body.

34. The tubular unit of claim 33, further comprising a plurality of said support rings which are spaced apart from each other along a lengthwise direction of said flexible tubular body.
